(19) United States
(12) Patent Application Publication
(10) Pub. No.: US 2005/0228250 A1
(43) Pub. Date: Oct. 13, 2005

(54) System and method for visualization and navigation of three-dimensional medical images

(76) Inventors: Ingmar Bitter, Rockville, MD (US); Wei Li, East Brunswick, NJ (US); Michael Meissner, Port Jefferson, NY (US); Frank C. Dachille, Amityville, NY (US); Soren Grimm, Wurmlingen (DE); George Economos, Bayport, NY (US)

Correspondence Address: F. CHAU & ASSOCIATES, LLC, 130 WOODBURY ROAD, WOODBURY, NY, 11797, US

(21) Appl. No.: 10/496,435

(22) PCT Filed: Nov. 21, 2002
(86) PCT No.: PCT/US02/37397

§ 371 Date: May 20, 2004

Related U.S. Application Data
(60) Provisional application No. 60/331,799, filed on Nov. 21, 2001.

(51) Int. Cl. A61B 5/05

(52) U.S. Cl. 600/407

(57) ABSTRACT

A user interface (90) comprises an image area that is divided into a plurality of views for viewing corresponding 2-dimensional and 3-dimensional images of an anatomical region. Tool control panes (95-101) can be simultaneously opened and accessible. The segmentation pane (98) enables automatic segmentation of components of a displayed image within a user-specified intensity range or based on a predetermined intensity

### CROSS-REFERENCE TO RELATED APPLICATION

This application claims priority to U.S. Provisional Application No. 60/331,799, filed on Nov. 21, 2001, which is fully incorporated herein by reference.

### COPYRIGHT NOTICE

A portion of the disclosure of this patent document contains material which is subject to copyright protection. The copyright owner has no objection to the facsimile reproduction by any one of the patent document or the patent disclosure, as it appears in the patent and Trademark Office patent file or records, but otherwise reserves all copyright rights whatsoever.

### TECHNICAL FIELD OF THE INVENTION

The present invention relates generally to systems and methods for aiding in medical diagnosis and evaluation of internal organs (e.g., colon, heart, etc.) More specifically, the invention relates to a 3D visualization (v3D) system and method for assisting in medical diagnosis and evaluation of internal organs by enabling visualization and navigation of complex 2D or 3D data models of internal organs, and other components, which models are generated from 2D image datasets produced by a medical imaging acquisition device (e.g., CT, MRI, etc.).

### BACKGROUND

Various systems and methods have been developed to enable two-dimensional (“2D”) visualization of human organs and other components by radiologists and physicians for diagnosis and formulation of treatment strategies. Such systems and methods include, for example, x-ray CT (Computed Tomography), MRI (Magnetic Resonance Imaging), ultrasound, PET (Positron Emission Tomography) and SPECT (Single Photon Emission Computed Tomography).

Radiologists and other specialists have historically been trained to analyze scan data consisting of two-dimensional slices. Three-Dimensional (3D) data can be derived from a series of 2D views taken from different angles or positions. These views are sometimes referred to as “slices” of the actual three-dimensional volume. Experienced radiologists and similarly trained personnel can often mentally correlate a series of 2D images derived from these data slices to obtain useful 3D information. However, while stacks of such slices may be useful for analysis, they do not provide an efficient or intuitive means to navigate through a virtual organ, especially one as tortuous and complex as the colon, or arteries. Indeed, there are many applications in which depth or 3D information is useful for diagnosis and formulation of treatment strategies. For example, when imaging blood vessels, cross-sections merely show slices through vessels, making it difficult to diagnose stenosis or other abnormalities.

### SUMMARY OF THE INVENTION

The present invention is directed to a systems and methods for visualization and navigation of complex 2D or 3D data models of internal organs, and other components, which models are generated from 2D image datasets produced by a medical imaging acquisition device (e.g., CT, MRI, etc.).

In one aspect of the invention, a user interface is provided for displaying medical images and enabling user interaction with the medical images. The User interface comprises an image area that is divided into a plurality of views for viewing corresponding 2-dimensional and 3-dimensional images of an anatomical region. The UI displays a plurality of tool control panes that enable user interaction with the images displayed in the views. The tool control panes can be simultaneously opened and accessible. The control panes comprise a segmentation pane having buttons that enable automatic segmentation of components of a displayed image within a user-specified intensity range or based on a predetermined intensity range (e.g. air, tissue, muscle, bone, etc.). A components pane provides a list of segmented components. The component pane comprises a tool button for locking a segmented component, wherein locking prevents the segmented component from being included in another segmented component during a segmentation process. The component pane comprises options for enabling a user to label a component, select a color in which the segmented component is displayed, select an opacity for a selected color of the segmented component, etc. An annotations pane comprises a tool that enables acquisition and display of statistics of a segmented component, e.g., an average image intensity, a minimum image intensity, a maximum intensity, standard deviation of intensity, volume, and any combination thereof.

In another aspect of the invention, the user interface displays icons representing containers for volume rendering settings, wherein volume rendering settings can be shared among a plurality of views or copied from one view into another view. The rendering settings that can be shared or copied between views include, e.g., volume data, segmentation data, a color map, window/level, a virtual camera for orientation of 3D views, 2D slice position, text annotations, position markers, direction markers, measurement annotations. The settings can be shared by, e.g., selecting a textual or graphical representation of the rendering setting and dragging the selected representation to a 2D or 3D view in which the selected representation is to be shared. Copying can be performed by selection of an additional key while dragging the selected setting in the view.

In another aspect of the invention, a user interface can display an active 2D slice in a 3D image to provide cross-correlation of the associated views. The 2D slice can be rendered in the 3D image with depth occlusion. The 2D slice an be rendered partially transparent in the 3D view. The 2D image can be rendered as colored shadow on a surface of an object in the 3D image.

These and other aspects, features and advantages of the present invention will become apparent from the following detailed description of preferred embodiments, which is to be read in connection with the accompanying drawings.

### BRIEF DESCRIPTION OF THE DRAWINGS

FIG. 1 is a diagram of a 3D imaging system according to an embodiment of the invention.

FIG. 2 is a flow diagram of a method for processing image data according to an embodiment of the invention

FIG. 3 is a flow diagram of a method for processing image data according to an embodiment of the invention.

FIG. 4 is a diagram illustrating user interface controls according to an embodiment of the invention.

FIGS. 5a and 5b are diagrams of user interfaces according to embodiments of the invention.

FIG. 6 is a diagram illustrating various layouts for 2D and 3D views in a user interface according to the invention.

FIG. 7 is a diagram illustrating a graphic framework of a visualization pane according to an embodiment of the invention.

FIG. 8 is a diagram illustrating a graphic framework of a segmentation pane according to an embodiment of the invention.

FIG. 9 is a diagram illustrating a graphic framework of a components pane according to an embodiment of the invention.

FIG. 10 is a diagram illustrating a graphic framework of an annotations pane according to an embodiment of the invention.

FIG. 11 is a diagram illustrating a graphic framework of a user preference window according to an embodiment of the invention.

FIGS. 12a-c are diagrams illustrating a method for displaying information in a 2D view according to an embodiment of the invention.

FIGS. 13a-c are diagrams illustrating graphic frameworks for 2D image tools and associated menu functions, according to embodiments of the invention.

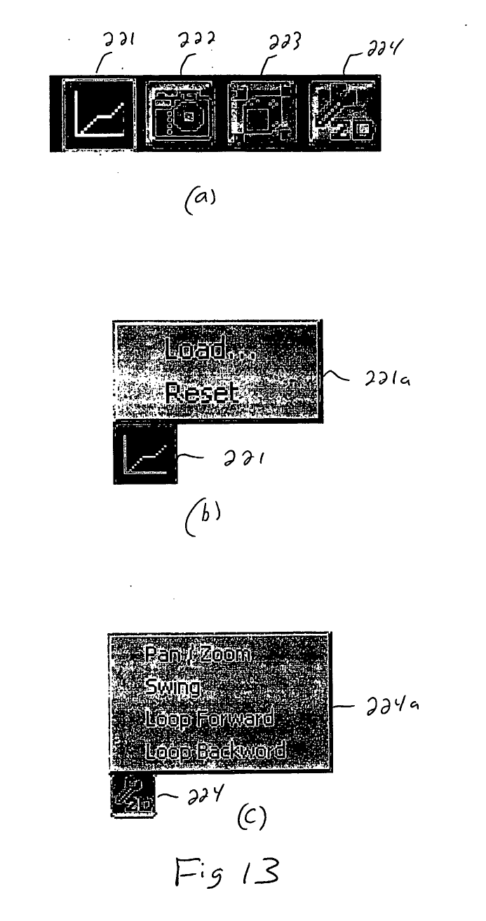

FIGS. 14a-d are diagrams illustrating graphic frameworks for 3D image tools and associated menu functions, according to embodiments of the invention.

FIG. 15 is a diagram illustrating a method for sharing volume rendering parameters between different views, according to the invention.

FIGS. 16a-b are diagrams illustrating a method for recording annotations according to embodiments of the invention.

FIG. 17 illustrates various measurements and annotations according to the invention.

FIG. 18 is a diagram illustrating a method for displaying control panes according to the invention.

FIGS. 19a-b are diagrams illustrating a method of correlating 2D and 3D images according to an embodiment of the invention.

### DETAILED DESCRIPTION OF PREFERRED EMBODIMENTS

The present invention is directed to medical imaging systems and methods for assisting in medical diagnosis and evaluation of a patient. Imaging systems and methods according to preferred embodiments of the invention enable visualization and navigation of complex 2D and 3D models of internal organs, and other components, which are generated from 2D image datasets generated by a medical imaging acquisition device (e.g., MRI, CT, etc.).

It is to be understood that the systems and methods described herein in accordance with the present invention may be implemented in various forms of hardware, software, firmware, special purpose processors, or a combination thereof. Preferably, the present invention is implemented in software as an application comprising program instructions that are tangibly embodied on one or more program storage devices (e.g., magnetic floppy disk, RAM, CD Rom, ROM and flash memory), and executable by any device or machine comprising suitable architecture.

It is to be further understood that since the constituent system modules and method steps depicted in the accompanying Figures are preferably implemented in software, the actual connection between the system components (or the flow of the process steps) may differ depending upon the manner in which the present invention is programmed. Given the teachings herein, one of ordinary skill in the related art will be able to contemplate these and similar implementations or configurations of the present invention.

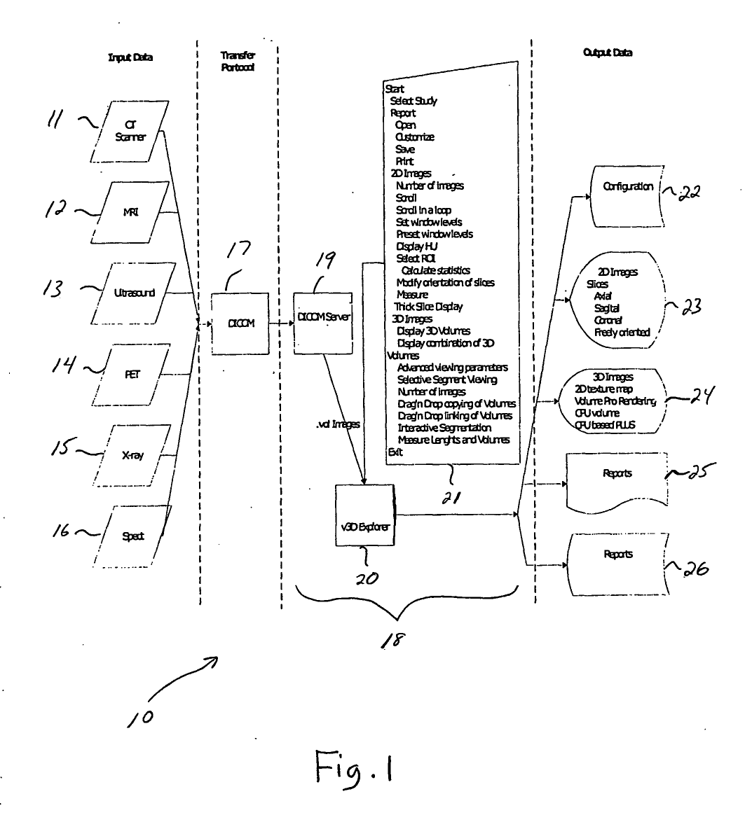

FIG. 1 is a diagram of an imaging system according to an embodiment of the present invention. The imaging system (10) comprises a 3D image processing application tool (18) which receives 2D image datasets generated by one of various medical image acquisition devices, which are formatted in DICOM format by DICOM module (17). For instance, the 2D image datasets comprise a CT (Computed Tomography) dataset (11) (e.g., Electron-Beam Computed Tomography (EBCT), Multi-Slice Computed Tomography (MSCT), etc.), an MRI (Magnetic Resonance Imaging) dataset (12), an ultrasound dataset (13), a PET (Positron Tomography) dataset (14), an X-ray dataset (15) and SPECT (Single Photon Emission Computed Tomography) dataset (16). It is to be understood that the system (19) can be used to interpret any DICOM formatted data.

The 3D imaging application (18) comprises a 3D imaging tool (20) referred to herein as the “V3D Explorer” and a library (21) comprising a plurality of functions that are used by the tool. The V3D Explorer (20) is a heterogeneous image-processing tool that is used for viewing selected anatomical organs to evaluate internal abnormalities. With the V3D Explorer, a user can display 2D images and construct a 3D model of any organ, e.g., liver, lungs, heart, brain colon, etc. The V3D Explorer specifies attributes of the patient area of interest, and an associated UI offers access to custom tools for the module. The V3D Explorer provides a UI for the user to produce a novel, rotatable 3D model of an anatomical area of interest from an internal or external vantage point. The UI provides access points to menus, buttons, slider bars, checkboxes, views of the electronic model and 2D patient slices of the patient study. The user interface is interactive and mouse driven, although keyboard shortcuts are available to the user to issue computer commands.

The output of the 3D imaging tool (20) comprises configuration data (22) that can be stored in memory, 2D images (23) and 3D images (24) that are rendered and displayed, and reports comprising printed reports (25) (fax, etc.) and reports (26) that are stored in memory.

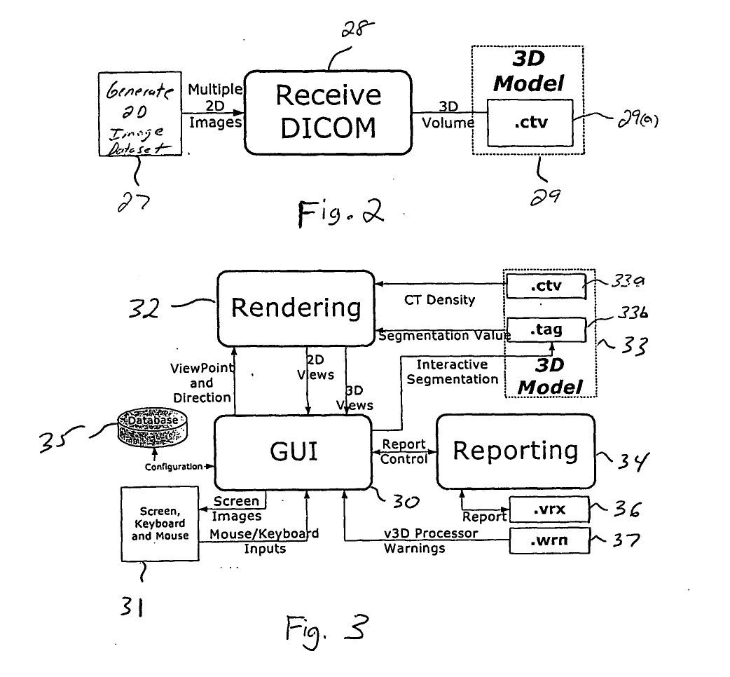

FIG. 2 is a diagram illustrating data processing flow in the system (10) of FIG. 1 according to one aspect of the invention. A medical imaging device generates a 2D image dataset comprising a plurality of 2D DICOM-formatted images (slices) of a particular anatomical area of interest (step 27). The 3D imaging system (18) receives the DICOM-formatted 2D images (step 28) and then generates an initial 3D model (step 29) from a CT volume dataset derived from the 2D slices using known techniques. A .ctv file (29a) denotes the original 3D image data is used for constructing a 3D volumetric model, which preferably comprises a 3D array of CT densities stored in a linear array.

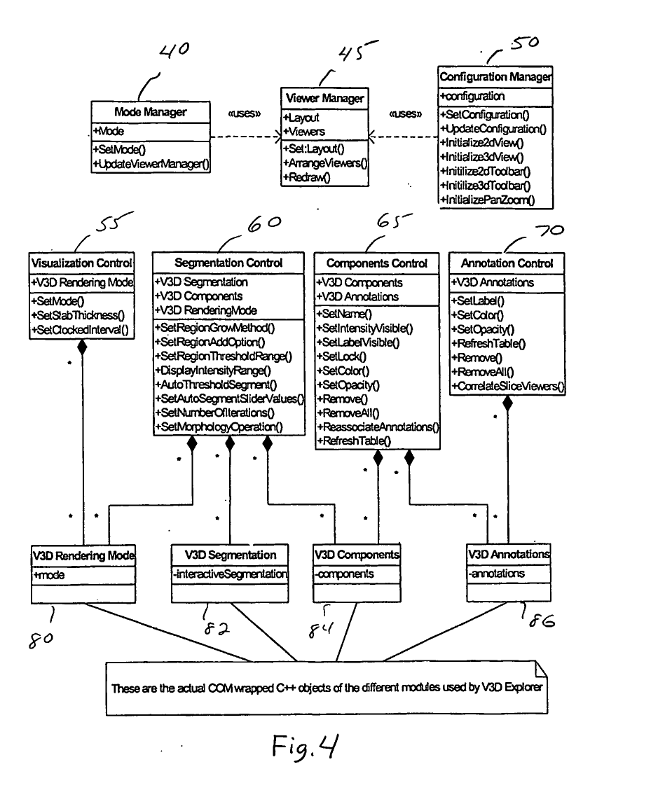

FIG. 3 is a diagram illustrating data processing flow in the 3D imaging system (18) of FIG. 1 according to one aspect of the invention. In particular, FIG. 3 illustrates data flow and I/O events between various modules comprising the V3D Explorer module (20), such as a GUI module (30), Rendering module (32) and Reporting module (34). Various I/O events are sent between the GUI module (30) and peripheral components (31) such as a computer screen, keyboard and mouse. The GUI module (30) receives input events (mouse clicks, keyboard inputs, etc.) to execute various functions such as interactive manipulation (e.g., artery selection) of a 3D model (33).

The GUI module (30) receives and stores configuration data from database (35). The configuration data comprises meta-data for various patient studies to enable a stored patient study to be reviewed for reference and follow-up evaluation of patient response treatment. The database (35) further comprises initialization parameters (e.g., default or user preferences), which are accessed by the GUI (30) for performing various functions. The rendering module (32) comprises one or more suitable 2D/3D renderer modules for providing different types of image rendering routines. The renderer modules (software components) offer classes for displays of orthographic MPR images and 3D images. The rendering module (32) provides 2D views and 3D views to the GUI module (30) which displays such views as images on a computer screen. The 2D views comprise representations of 2D planer views of the dataset including a transverse view (i.e., a 2D planar view aligned along the Z-axis of the volume (direction that scans are taken)), a sagittal view (i.e., a 2D planar view aligned along the Y-axis of the volume) and a Coronal view (i.e., a 2D planar view aligned along the X-axis of the volume). The 3D views represent 3D images of the dataset. Preferably, the 2D renderers provide adjustment of window/level, assignment of color components, scrolling, measurements, panning zooming, information display, and the ability to provide snapshots. Preferably, the 3D renderers provide rapid display of opaque and transparent endoluminal and exterior images, accurate measurements, interactive lighting, superimposed centerline display, superimposed locating information, and the ability to provide snapshots.

The rendering module (32) presents 3D views of the 3D model (33) to the GUI module (30) based on the viewpoint and direction parameters (i.e., current viewing geometry used for 3D rendering) received from the GUI module (30). The 3D model (33) comprises an original CT volume dataset (33a) and a tag volume (33b) which comprising a volumetric dataset comprising a volume of segmentation tags that identify which voxels are assigned to which segmented components. Preferably, the tag volume (33b) contains an integer value for each voxel that is part of some known (segmented region) as generated by user interaction with a displayed 3D image (all voxels that are unknown are given a value of zero). When rendering an image, the rendering module (32) overlays the original volume dataset (33a) with the tag volume (33b).

As explained in more detail below, the V3D Explorer (20) can be used to interpret any DICOM formatted data. Using the V3D Explorer (20), a trained physician can interactively detect, view, measure and report on various internal abnormalities in selected organs as displayed graphically on a personal computer (PC) workstation. The V3D Explorer (20) handles 2D-3D correlation as well as other enhancement techniques, such as measuring an anomaly. The V3D Explorer (20) can be used to detect abnormalities in 2D images or the 3D volume generated model of the organ. Quantitative measurements can be made, for both size and volume, and these can be tracked over time to analyze and display the change(s) in abnormalities. The V3D Explorer (20) allows a user to pre-set configurable personal preferences for ease and speed of use.

An imaging system according to the invention preferably comprises an annotation module (or measuring module) provides a set of measurement and annotation classes. The measurement classes create, visualize and adjust linear, ROI, angle, volumetric and curvilinear measurements on orthogonal, oblique and curved MPR slice images and 3D rendered images. The annotation classes can be used to annotate any part of an image, using shapes such as arrow or a point in space. The annotation module calculates and displays the measurements and the statistics related to each measurement that is being drawn. The measurements are stored as a global list which may be used by all views. In addition, an imaging system according to the invention comprises a an interactive Segmentation module provides a function for classifying and labeling medical volumetric data. The segmentation module comprises functions that allow the user to create, visualize and adjust the segmentation of any region within orthogonal, oblique, curved MPR slice image and 3D rendered images. The segmentation module produces volume data to allow display of the segmentation results. The segmentation module is interoperable with the annotation (measuring) module to provide width, height, length volume, average, max, std deviation, etc of a segmented region.

The V3D Explorer provides a plurality of features and functions for viewing, navigation, and manipulating both the 2D images and the 3D volumetric model. Such functions and features include, for example, 2D features such as (i) window/level presets with mouse adjustment (ii) 2D panning and zooming; (iii) the ability to measure distances, angles and Region of Interest (ROI) areas, and display statistics on 2D view; and (iv) navigation through 2D slices. The 3D volume model image provides features such as (i) full volume viewing (exterior view); (ii) thin slab viewing in the 2D images; and (iii) 3D rotation, panning and zooming capability.

Further, the V3D Explorer simplifies the examination process by supplying various Window/Level and Color mapping (transfer function) presets to set the V3D for standard needs, such as (i) Bone, Lung, and other organ Window/Level presets; (ii) scanner-specific presets (CT, MRI, etc.); (iii) color-coding with grayscale presets, etc.

The V3D Explorer allows a user to: (i) set specific volume rendering parameters; (ii) perform 2D measurements of linear distances and volumes, including statistics (such as standard deviation) associated with the measurements; (iii) provide an accurate assessment of abnormalities; (iv) show correlations in the 2D slice positions; and (v) localize related information in 2D and 3D images quickly and efficiently.

The V3D Explorer displays 2D orthogonal images of individual patient slices that are scrollable with the mouse wheel, and automatically tags (colorizes) voxels within a user-defined intensity range for identification.

Other novel features and functions provided by the V3D Explorer include (i) a user-friendly Window Level and Colormap editor, wherein each viewer can adjust to the user's specific functions or Window/Level parameters for the best view of an abnormality; (ii) the sharing of settings among multiple viewers, such as volume, camera angle (viewpoint), window/level, transfer function, components; (iii) multiple tool controls that are visible and accessible simultaneously; and (iv) intuitive interactive segmentation, which provides (i) single click region growing; (ii) single click classification into similar tissue groups; and (iii) labeling, coloring, and selectively displaying components, which provides a convenient way to arbitrarily combine the display of different components.

In a preferred embodiment of the invention, the V3D Explorer module comprises GUI controls such as: (i) Viewer Manager for managing the individual viewers where data is rendered; (iii) Configuration Manager Control, for setting up the different number and alignment of viewers; (iv) Patient & Session Control, for displaying the patient and session information; (v) Visualization Control, for handling the rendering mode input parameters; (vi) Segmentation Control, for handling the segmentation input parameters; (vii) Components Control, for displaying the components and handling the input parameters; (viii) Annotations Control, for displaying the annotations and handling the input parameters; and (ix) Colormap Control, for displaying the window/level or color map and handling the input parameters.

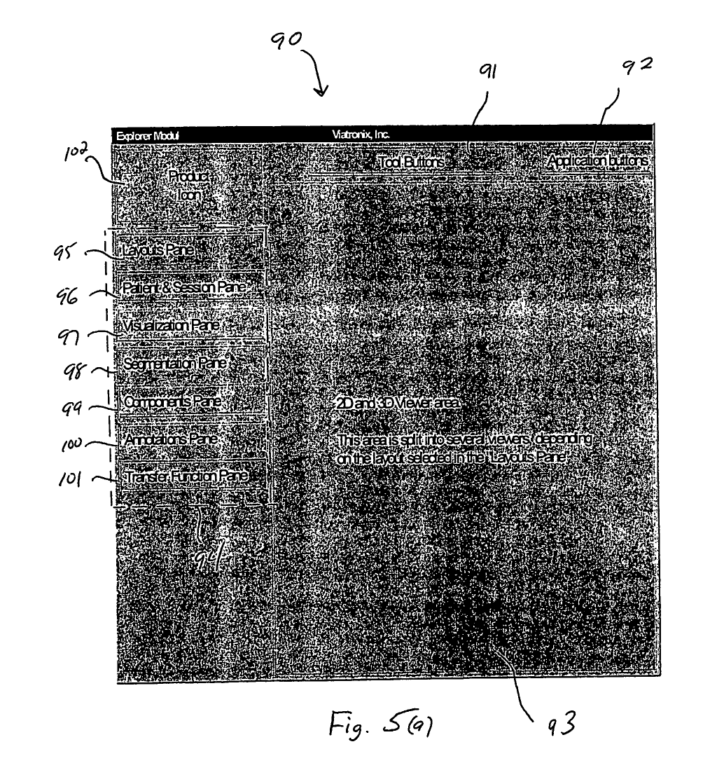

FIG. 4 illustrates the relation and access paths between various GUI controls of the Explorer module (20) (FIG. 1) according to one embodiment of the invention. In the following, all depicted functions that are not self explanatory will be explained, e.g. self explanatory is SetName( ) which obviously will pass a name in form of a string and store it as member.

A Viewer Manager control (45) comprises functions such as:

SetLayout( ), which takes an enumeration value encoding the requested layout of viewers on the screen. This only denotes the viewer layout on the screen but not what renderers or manipulators go in;
ArrangeViewers( ), which reorganizes the screen/layout based on the current layout. For each window, a viewer is created and initialized; and
Redraw( ), which issues a redraw on all currently active viewers. A Configuration Manager control (50) provide function such as:
SetConfiguration( ), which takes an enumeration value encoding the configuration denoting which manipulator and renderer needs to go into each of the viewers in the layout;
UpdateConfiguration( ), which applies the selected configuration and issues the initialization of the individual viewers;
Initialize2dView( ), which takes as parameter the MPR orientation which can be axial, coronal, or sagittal. It adds all default manipulators and renderers that belong to a default MPR view such as MPR renderer, annotation renderer, overlay renderer, manipulator for moving the slice, manipulator for current voxel, and manipulator for slice shadow;
Initialize3dView( ), which adds all default manipulators and renderers that belong to a default three dimensional view such as 3D renderer, annotation renderer, overlay renderer, and manipulator for camera manipulation;
Initialize2dToolbar( ), which adds all default toolbar buttons for a MIP view which are color map, orientation, 2D tools, and snapshot.

Initialize2dToolbar( ), which adds all default toolbar buttons for a 3D view which are color map, orientation, 3D tools, and snapshot.

InitializePanZoom( ), which initializes the pan/zoom or orientation cube window with the corresponding renderers and manipulators.

A Visualization Control (55) provides functions such as:

SetMode( ), SetSlabthickness( ) and SetClockedInterval, which functions are self-explanatory.

A Segmentation Control (60) provides functions such as:

SetRegionGrowMethod( ), which takes an enumeration type and sets the method to region or sample based;
SetRegionAddOption( ), which takes an enumeration type and sets the option to “new” or “add”;
SetRegionThresholdRange( ), which takes as input two values that represent the lower and upper bound of the voxel values to be considered;
DisplayIntensityRange( ), which changes the rendering mode to give a feedback to the users which of the currently visible voxels belong to this range;
AutoThresholdSegmentso, which issues segmentation on the entire dataset and assigns a new component index to all voxels that belong to the currently selected value range. This creates a component and needs to add this to the component table by notifying a components control (65);
SetAutoSegmentSliderValues( ), which takes as input two values that represent the lower and upper bound of the voxel values to be considered for auto segmentation, overwriting the defaults; and
SetMorphologyOperation( ), which takes an enumeration type and selects either “open”, “close”, “erode”, or “dilate”.
   A Components Control (65) provides functions such as:
SetIntensityVisible( ), which takes the index of the currently selected component and toggles the current visible flag.
SetLabelVisible( ), which takes the index of the currently selected component and toggles the current label flag;
SetLock( ), which takes the index of the currently selected component and toggles the current lock flag;
SetColor( ), which takes a RGB color and sets the member to hold this color;
SetOpacity( ), which takes an opacity and sets the member to holds this opacity;
Remove( ), which takes the index of the currently selected component and removes it from the list of components;
RemoveAll( ), which clears the list of components in one run allowing to optimize it because no update of any internal structure is needed as in removing each component at a time;
ReassociateAnnotations( ), which is called after removing one or more components to see if there was any annotation related to any of the removed components. If yes, this annotation can be removed as well; and
RefreshTable( ), which is called to redraw the table after any type of modification. An Annotation Control (70) comprises functions such as:
SetLabel( ), which takes a string and sets the member to hold this label string.
SetColor( ), which takes a RGB color and sets the member to hold this color.
SetOpacity( ), which takes an opacity and sets the member to holds this opacity.
RefreshTable( ), which is called to redraw the table after any type of modification.
Remove( ), which takes the index of the currently selected annotation and removes it from the list of annotations.;
RemoveAll( ), which clears the list of annotations in one run allowing to optimize it because no update of any internal structure is needed as in removing each annotation at a time; and
CorrelateSliceViewers( ), which goes through all v3D environments and for the ones that are 2D views, it sets the currently display MPR slice to the one in which the currently selected annotation resides.

The role of each of the above controls and functions will become more apparent based on the discussion below.

Graphical User Interface—V3D Explorer

The following section describes GUIs for a V3D Explorer application according to preferred embodiments of the invention. As noted above, a GUI (or User Interface (UI) or “interface”) provides a working environment of the V3D Explorer. In general, a GUI provides access points to menus, buttons, slider bars, checkboxes, views of the electronic model and 2D patient slices of the patient study. Preferably, the user interface is interactive and mouse driven, although keyboard shortcuts are available to the user to issue computer commands. The V3D Explorer's intuitive interface uses a standard computer keyboard and mouse for inputs. The user interface displays orthogonal and multiplanar reformatted (MPR) images, allowing radiologists to work in a familiar environment. Along with these images is a volumetric 3D model of the organ or area of interest. Buttons and menus are used to input commands and selections.

A patient study file can be opened using V3D Explorer. A patient study comprises data containing 2D slice data, and after the first evaluation by the V3D Explorer it also contains a non-contrast 3D model with labels and components. A “Session” as used herein refers to a saved patient study dataset including all the annotations, components and visualization parameters.

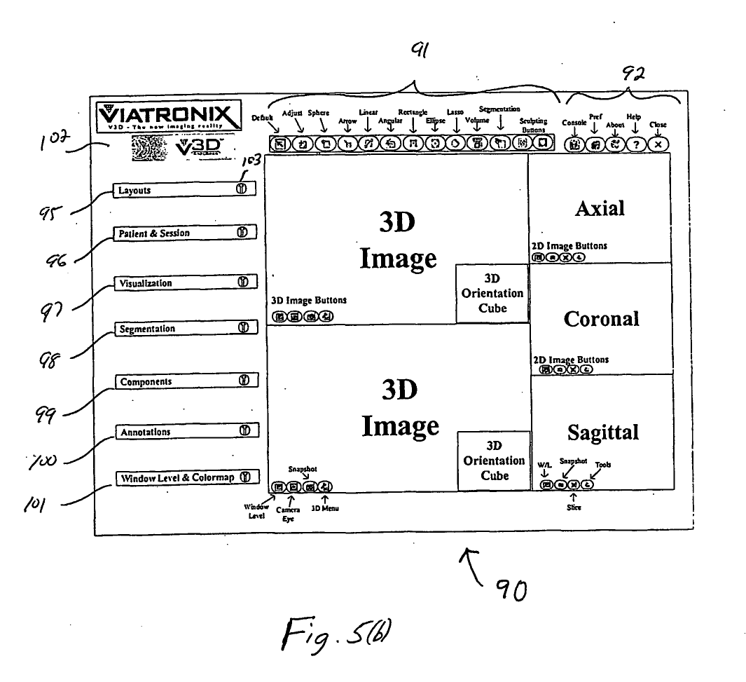

FIG. 5a is an exemplary diagram of a GUI according to an embodiment of the invention, which illustrates a general layout of a GUI. In general, a GUI (90) comprises different areas for displaying tool buttons (91) and application buttons (92). The GUI (90) further comprises an image area (93) (or 2D/3D viewer area) and an information area (94). In addition, a product icon area (102) can be included to display a product icon in text and color of the v3D Explorer Module product. FIG. 5(b) is an exemplary diagram of a GUI according to another embodiment of the invention, which illustrates a more specific layout of a GUI based on the framework shown in FIG. 5(a).

The image area (93) displays one or more “views” in a certain arrangement depending on the selected layout configuration. Each “view” comprises an area for displaying an image (3D or 2D), displaying pan/zoom or orientation, and an area for displaying tools (see, FIG. 5b). The GUI (90) allows the user to change views to present various 2D/3D configurations. The image area (93) is split into several views, depending on the layout selected in a “Layouts” pane (95). The image area (93) contains the 2D images (slices) contained in a selected patient studies and the 3D images needed to perform various examinations, in configurations defined by the Layout Pane (95). In the 2D images, for each cursor position (called a voxel), the V3D Explorer GUI can display the value of that position in Hounsfield Units (HU) or raw density values (when available).

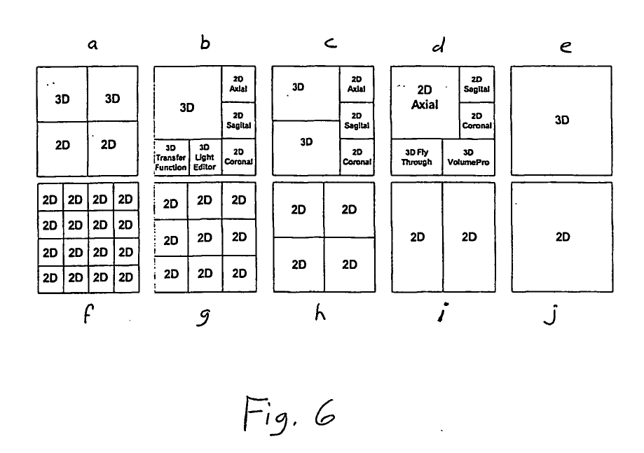

FIGS. 6(a)-(j) illustrate various image window configurations for presenting 2D or 3D views, or combinations of 2D and 3D views in the image area (93). The V3D Explorer GUI (90) can display various types of images including, a cross-sectional image, three 2D orthogonal slices (axial, sagittal and coronal) and a rotatable 3D virtual mode of the organ of interest. The 2D orthogonal slices are used for orientation, contextual information and conventional selection of specific regions. The external 3D image of the anatomical area provides a translucent view that can be rotated in all three axes. Anatomical positional markers can be used to show where the current 2D view is located in a correlated 3D view. The V3D Explorer has many arrangements of 2D slice images—multiplanar reformatted (MPR) images, as well as the volumetric 3D model image. In the nine-frame layout shown in FIG. 6(g), for example, the 2D slices can be linked by column, letting the user view axial, coronal and sagittal side-by-side, and to view different slices in different views. Each frame can be advanced to different slices.

FIG. 6(f) illustrates 2D slice images shown in sixteen-frame format, which is a customary method of radiologists and clinicians for viewing 2D slices. FIG. 5(b) illustrates a view configuration as depicted in FIG. 6(c), where different rendering techniques may be applied in different 3D views.

Referring again to FIG. 5(a), the information area (94) of the GUI (90) comprises a plurality of Information Panes (95-101) that provide specific features, controls and information. The GUI (90) comprises a pane for each of the GUI controls described above with reference to FIG. 4. More specifically, in a preferred embodiment of the invention, the GUI (90) comprises a layouts pane (95), a patient & session pane (96), a visualization pane (97), a segmentation pane (98), a components pane (99), an annotations pane (100) and a colormap pane (101) (or Window Level & Colormap pane). As shown in FIG. 5(b), each pane comprises a pane expansion selector (103) (expansion arrow) on the top right to expand and/or contract the pane. Pressing the corresponding arrow (103) toggles the display of the pane. The application is able to show multiple pane open and accessible at the same time. This is different from the traditional tabbed views that allow access to only one pane at the time.

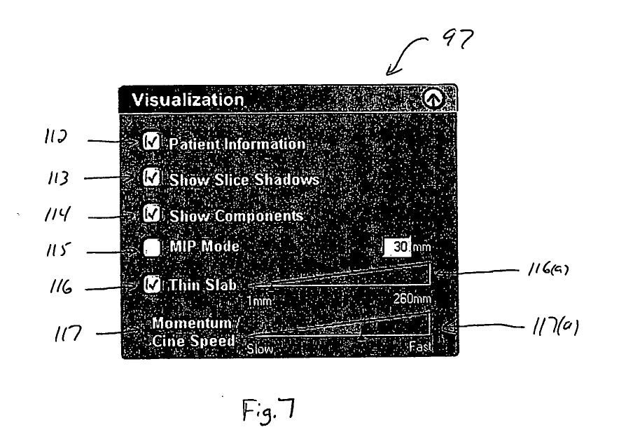

FIG. 7 is a diagram illustrating a graphic framework for the Visualization pane (97) according to an embodiment of the invention. The Visualization pane (97) allows a user to control the way in which V3D Explorer application displays certain features on the images, such as “Patient Information”. To select certain features (112-117), a check box is included in the control pane (97) which can be selected by the user to activate certain features within the pane. Clicking on a box next to a feature will place a checkmark in the box and activate that feature and clicking again will remove the check and deactivate the feature.

As shown in FIG. 7, various features controlled through checking the boxes in the Visualization pane (97) include: Patient Information (112) (which displays the patient data on the 2D and 3D slice images, when checked), Show Slice Shadows (113), Show Components (114); Maximum Intensity Projection (MIP) Mode (115), Thin Slab (116) (Sliding Thin Slab), and Momentum/Cine Speed (117). The “Show Slice Shadows” feature (113) allows a user to view the intersection between a selected image and other 2D slices and 3D images displayed in image area (93). This feature enables correlation of the different 2D/3D views. These “markers”, which are preferably colored shadows (in the endoluminal views) or slice planes, indicate the current position of a 2D slices relative to the selected image (3D, axial, coronal, etc.). The “shadow” of other selected slice(s) can also be made visible if desired. Using the feature (113) enables the user to show the various intersection planes as they correlate the location an area of interest in the 2D and 3D images.

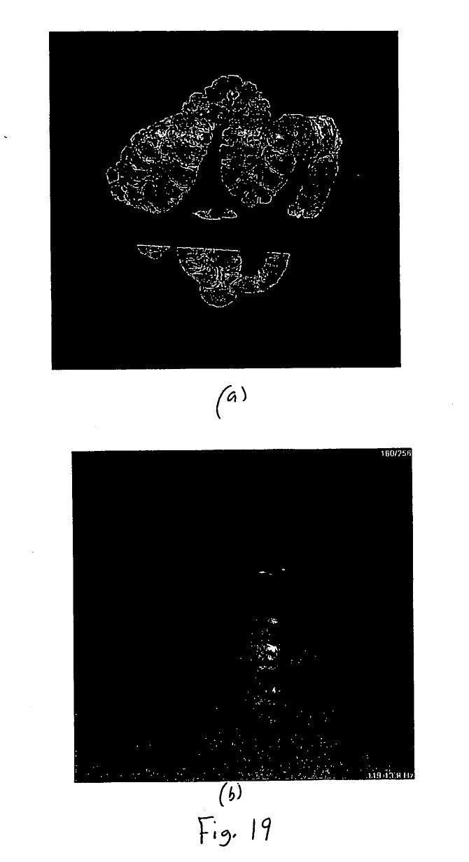

For instance, FIGS. 19a and 19b illustrate a 2D slice is embedded in a 3D view. With this method, it is preferred that proper depth occlusion allows parts of the slice to occlude parts of the 3D object and vice versa (the one in front is visible). If the plain or the object is partially transparent then the occlusion is only partial as well and the other object can be seen partially through the one in front.

The “Show Components” feature (114) can be selected to display “components” that are generated by the user (via segmentation) during the examination. The term “component” as used herein refers to an isolated region or area that is selected by a user on a 2D slice image or the 3D image using any of User Tools Buttons (91) (FIGS. 5a, 5b) described herein. As explained in further detail below, a user can assign a color to a component, change the clarity, and “lock” the component when finished. By deactivating the “Show Components” feature (114) (removing the check mark), the user can view the original intensity volume of a displayed image, making the components invisible.

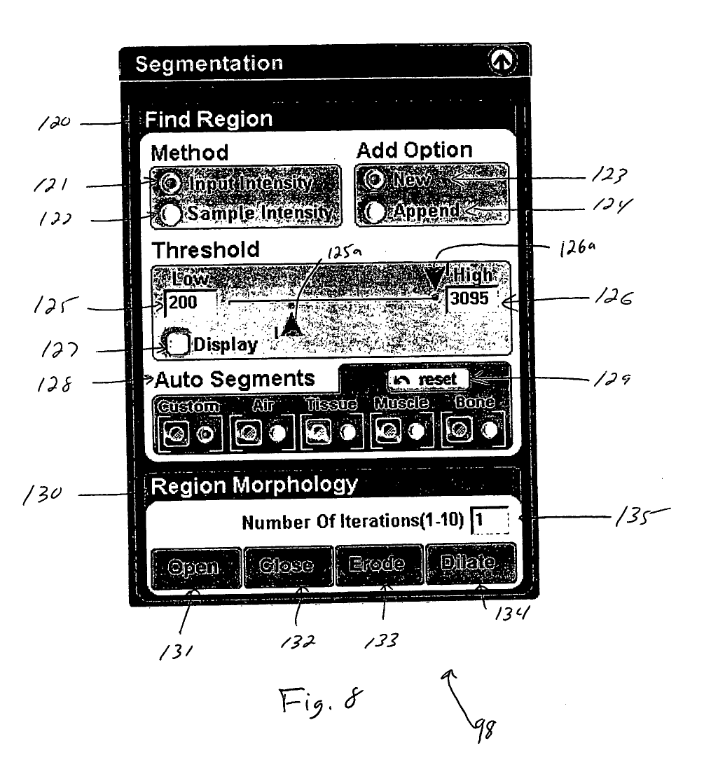

FIG. 8 is a diagram illustrating a graphic framework of a segmentation pane according to an embodiment of the invention. The segmentation pane (98) allows a user to select one of various Automatic Segmentation features (128). More specifically, an Auto Segments section (128) of the Segmentation pane (98) allows the user to preset buttons to automatically segment specific types of areas or organs, such as air, tissue muscle, bone. Just as the V3D Explorer offers preset window/level values associated with certain anatomical areas, there are also preset density values already loaded into the application, plus a Custom setting where the user can store desired preset density values. More specifically, in a preferred embodiment, the V3D Explorer provides a plurality of color-coded presets for the most commonly used segmentation areas: Air (e.g., blue), Tissue (e.g., orange), Muscle (e.g., red) and Bone (e.g., brown), and one Custom (e.g., green) setting, that uses the current threshold values. When the user selects one of the buttons of the Auto Segments (128), the areas will segment automatically and take on the color of the buttons (e.g., Green for Custom setting, Blue for Air, Yellow for Tissue, Red for Muscle and Brown for Bone.) If the user changes the threshold values, the user can select a Reset button (129) to return the segmentation values to their original numbers.

The V3D Explorer uses timesaving Morphological Processing techniques, such as Dilation and Erosion, for dexterous control of the form and structure of anatomical image components. More specifically, the Segmentation pane (98) comprises a Region Morphology area (130) comprising an open button (131), close button (132), erode button (133) and a dilate button (134). When a component is selected, it can be colorized, removed, and/or made to dilate. The Dilate button (134) accomplishes this by adding an additional layer, as an onion has layers, on top of the current outer boundary of the component. Each time the Dilate button (134) is selected, the component expands another layer, thus taking up more room on the image and removing any “fuzzy edge” effect caused by selecting the component. The Erode button (133), which provides a function opposite of the dilation operation, removes a layer from the outside boundary, as peeling an onion. Each time the Erode button (133) is selected, the component looses another layer and “shrinks,” requiring less space on the image. The user can select a number of iterations (135) for performing such functions (131-134).

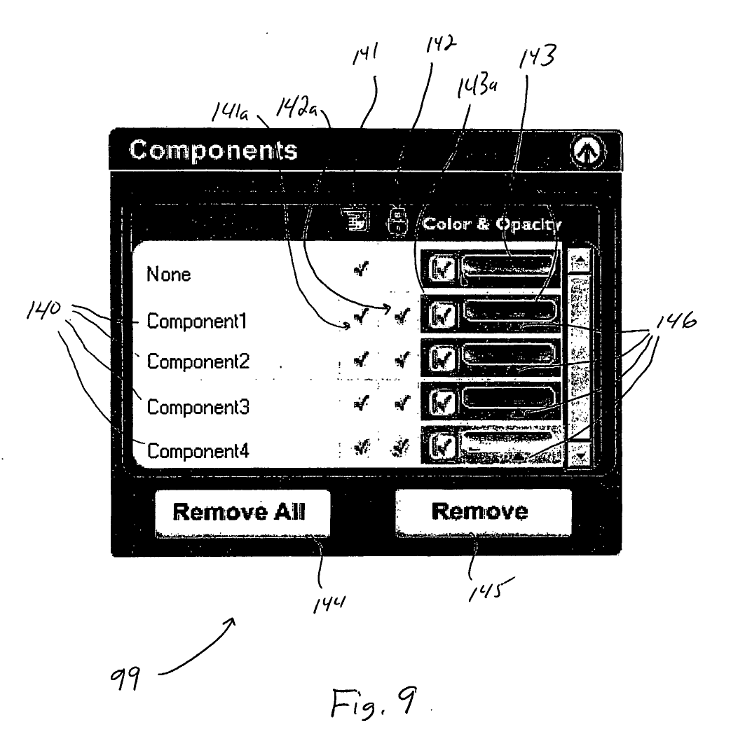

FIG. 9 is a diagram illustrating a graphic framework for the Components pane (99) according to an embodiment of the invention. The Components pane (99) provides a listing of all components (140) generated by the user (via the segmentation process). The component pane has an editable text field (140) for labeling each component. When a component (140) is selected, the V3D Explorer can fill the component with a color that is specified by the user and control the opacity/clarity (“see-through-ness”) of the component. For each component (140) listed in the Components pane (99), the user can select (check) an area (143a) to activate a color button (143) to show the color of the component and/or display intensities, select (check) a corresponding area (142a) to activate a lock button (142) to “lock” the component so it can not be modified, select a check button (143a) to use the color selected by the user, and/or select a button (143) to change the component's color or opacity (opaqueness) (using sliding bar 146). In a preferred embodiment, to change the color of a component, the color of any Component can be adjusted by double-clicking on the color strip bar to bring up the Windows® color pallet and selecting (or customizing) a new color. This method also applies to changing the color of Annotations (as described below). The user can remove all components by selecting button (144) or remove a selected component via button (145).

Further, there is a checkbox (141a) to select if the voxels associated with this component should be visible at all in any 2D or 3D view. There is a checkbox (142a) to lock (and un-lock) the component. When it is locked it will cause all further component operations (region finding, growing, sculpting) to exclude the voxels from this locked component. With this it is possible to keep a region grow from including regions that are not desired even through they have the same intensity range. For example, blood vessels that would be attached to bone in a simple region grow can be separated from the bone by first sculpting the bone, then locking it and then starting the region grow in the blood vessel.

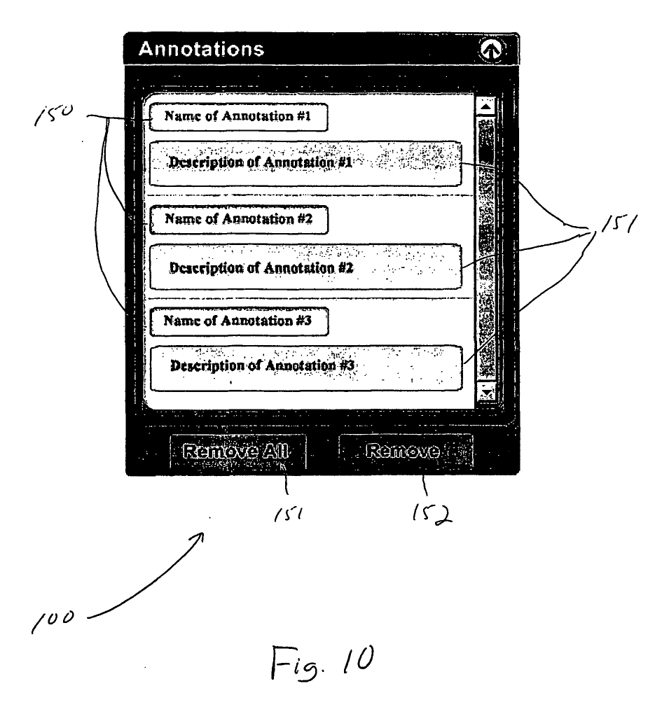

FIG. 10 is a diagram illustrating a graphic framework for the Annotations pane (100) according to an embodiment of the invention. The Annotation Pane (100) is the area where annotations and measurements are listed. In addition to the name (150) and description (151) of each annotation generated by the user, the annotations pane (100) also displays the type of annotation (e.g., what type of measurement) was made, and the user-specified color of the annotation. To remove an annotation, select it by clicking on it, and then hit the Remove button (152). To remove all the annotations, simple press the Remove All button (152).

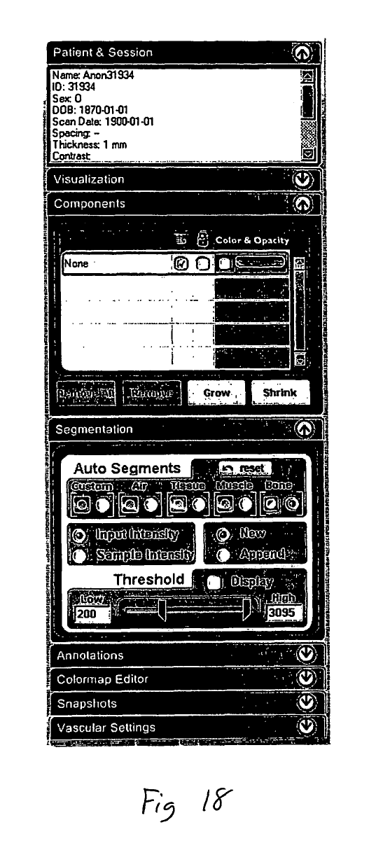

The panes (tool controls) are arranged as stacked rollout panes that can open individually. When all of them are closed they occupy only very little screen space and all available control panes are visible. When a pane is opened it “rolls out” pushes the re panes below further down such that all pane headings are still visible, but now the content of the open pane is visible as well. As long as there still is screen space available additional panes can be opened in the same manner. This is shown in FIG. 18. In addition, selecting one function can activate related panes. For example, selecting the find region mode automatically opens the segmentation pane and the components pane, as these are the ones most likely to be accessed when the user wants to find a region.

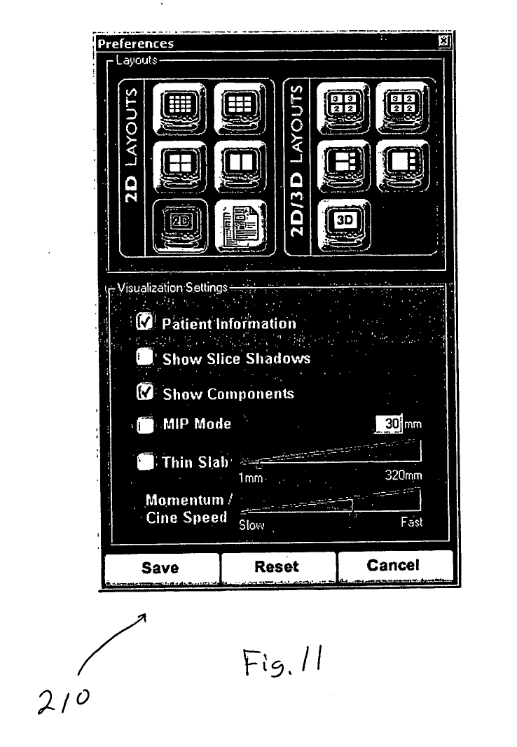

With the V3D Explorer application, the user can save a session with a patient study dataset. If there is a session stored for a given patient study that the user is opening, the V3D Explorer will ask if the user wants to open the session already stored or start a new session. It is to be understood that saving a session does not change the patient study dataset, only the visualization of the data. When the user activates the “close” button (tool bar 92, FIG. 5b), the V3D Explorer will ask if the user wishes to save the current session. If the user answers yes, the session will be saved using the current patient study file name. Answering No will close the application with no session saved. The “Help” button activates an interactive Help Application (which is beyond the scope of this application). The “Preferences” button provides the functionality to set user-specific parameters for layouts and Visualization Settings. The Preferences box also monitors the current Window/Level values and the Cine Speed. FIG. 11 illustrates a Preferences Button Display Window (210) according to an embodiment of the invention. In this window, the user can set the layout configuration of the GUI.

As noted above, the 2D/3D Renderer modules offer classes for displaying orthographic MPR, oblique MPR, and curved MPR images. The 2D renderer module is responsible for handling the input, output and manipulation of 2-dimensional views of volumetric datasets including three orthogonal images and the cross sectional images. Further, the 2D renderer module provides adjustment of window/level, assignment of color components, scrolling through sequential images, measurements (linear, ROI), panning, zooming of the slice information, information display, provide coherent positional and directional information with all other views in the system (image correlation) and the ability to provide snapshots.

The 3D renderer module is responsible for handling the input, output and manipulation of three-dimensional views of a volumetric dataset, and principally the endoluminal view. In particular, the 3D renderer module provides rapid display of opaque and transparent endoluminal and exterior images, accurate measurements of internal distances, interactive modification of lighting parameters, superimposed centerline display, superimposed display of the 2Ds slice location, and the ability to provide snapshots.

As noted above, the GUI of the V3D Explorer enables the user to select one of various image window configurations for displaying 2D and/or 3D images. For example, FIG. 5b illustrates an image window configuration that display two 3D views of an anatomical area of interest and three 2D views (axial, coronal, sagittal).

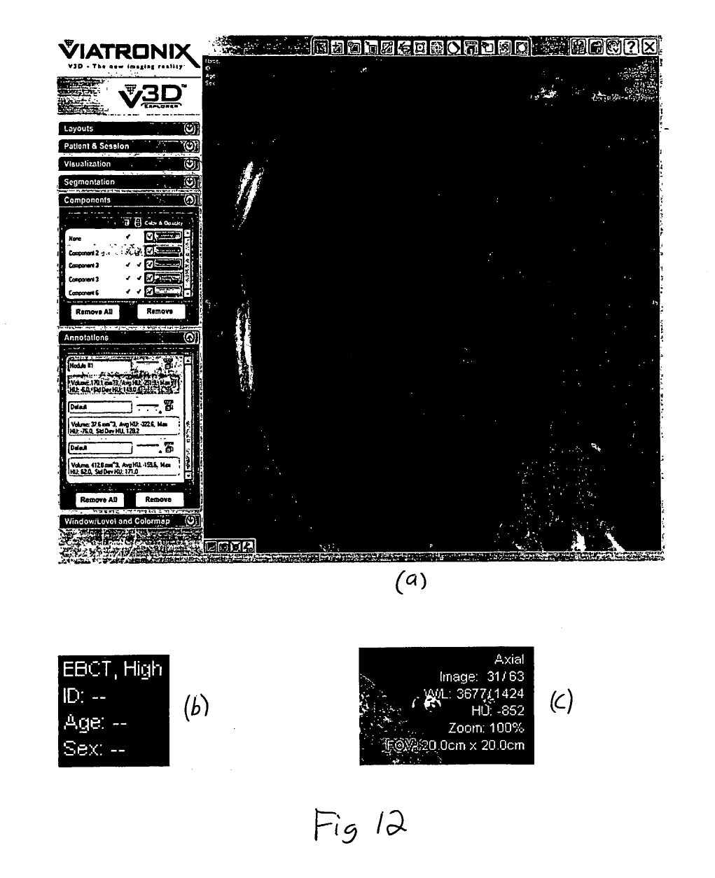

The V3D Explorer GUI provides various arrangements of 2D slice images, multiplanar reformatted (MPR) images, Axial, Sagittal and Coronal, for selection by the user, as well as the volumetric 3D model image. FIG. 12a is an exemplary diagram of GUI interface displaying a 2D Image showing a lung nodule. Patient and image information is overlaid on every 2D and 3D image displayed by the V3D Explorer. The user can active or deactivate the patient information display. On the left of the image is the Patient Information (FIG. 12b), and on the right is the image information: Slice (axial, sagittal, etc.), the Image Number, Window/Level (W/L), Hounsfield Unit (HU), Zoom Factor and Field of View (FOV).

The Window/Level of all 2D and 3D images is fully adjustable to permit greater control of the viewing image. Shown in the upper right of the image, the window level indicator shows the current Window and Level. The first number is the reading for the Window, and the second is for Level. To adjust the Window/Level use the right mouse button, dragging the mouse to increase or decrease the Window/Level. The V3D Explorer has the ability to regulate the contrast of the display in the 2D images. The Preset Window/Level feature offers customized settings to display specific window/level readings. Using these preset levels allows the user to isolate specific anatomical areas such as the lungs or the liver. The V3D Explorer preferably offers 10 preset window/level values associated with certain anatomical areas. These presets are defined by the specific HU values and can be accessed by, e.g., pressing the numerical keys (zero to nine) on the keyboard when the cursor is on a 2D image:

| Numerical | | Window, Level |
|---|---|---|
| Key | Anatomical Area | (in HUs) |
| 1 | ABDOMEN | 350, 40 |
| 2 | BONE | 100, 170 |
| 3 | CEREBRUM | 120, 40 |
| 4 | LIVER | 100, 70 |
| 5 | LUNG | −300, 2000 |
| 6 | HEAD | 80, 40 |
| 7 | PELVIS | 400, 40 |
| 8 | POSTERIOR FOSSA | 250, 80 |
| 9 | SUBDURAL | 150, 40 |
| 0 | CALCIUM | 1, 130 |

As shown in FIG. 12(c), under the window level indicator is the Hounsfield Unit (HU) reading for wherever the mouse pointer is positioned. Moving the mouse pointer around the image changes the HU reading as the mouse pointer crosses different density areas on the image. Raw density values are also displayed when available in the data.

In addition, the V3D Explorer displays the Field of View (FOV) below the Zoom Factor, which shoes the size of the magnified area shown in the image. The FOV decreases as the magnification increases

As discussed above, a Window/Level and Colormap function provides interactive control for advanced viewing parameters, allowing the user to manipulate an image by assigning window/level, hue and opaqueness to the various components defined by the user. The V3D Explorer includes more advanced presets than the ones mentioned above. These are available for loading through the Window/Level and Colormap Editor, and will make visualization and evaluation much easier by availing your session of already edited parameters for use in defining your components.

When a preset Transfer Function/Window Level is loaded. the V3D Explorer picks up the changes, reinterprets the 3D volume and redisplays it, all in an instant.

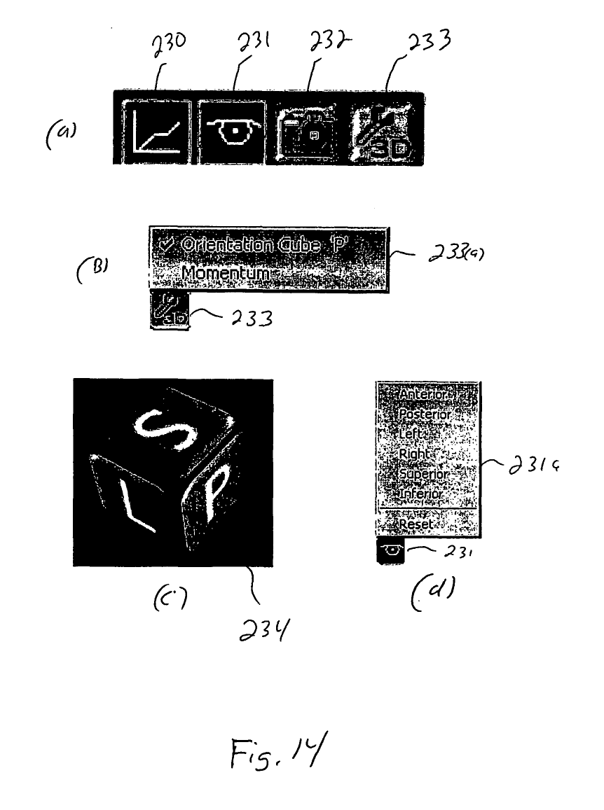

The user can load a preset parameter by going to the Window Level/Colormap button in the lower left of the image and using the Load option from a menu that is displayed when the button is selected. As shown in FIGS. 14 and 5b, in the lower left corner of the 3D image is a row of four (4) 3D image buttons. As more specifically shown in FIG. 14(a), these buttons include, for example, a Window Level/Colormap button 230, the Camera Eye Orientation button 231, the Snapshot button 232 and the 3D Menu button 233. The 3D image is rotatable in all three axes, allowing the user to orientate the 3D image for the best possible viewing. To rotate the image, the use would place the mouse pointer anywhere on the image and drag while holding the left mouse button down. The image will rotate accordingly. In the 3D image, the user can move the viewpoint closer or farther from the image by, e.g., placing the mouse pointer on the 3D image and scrolling the middle mouse wheel to move closer to or father back from the image.

As the user rotates and zooms the 3D image, the user could re-orientate the viewpoint back to the original position using a Camera Eye Orientation button 231 from the 3D image button row. Clicking on this button will display the Standard Views (Anterior, Posterior, Left, Right, Superior, Inferior), and the Reset option (as shown in FIG. 14(d). Selecting “reset” will return the 3D image to its original viewpoint. If there are two frames with the 3D images in them, and the user wants one frame to take on the viewpoint of the other, the user could simply click on the button and “drag and drop” it into the 3D frame that the user wants to change. When the user lets go of the left mouse button, the viewpoint in the second frame will match the other viewpoint.

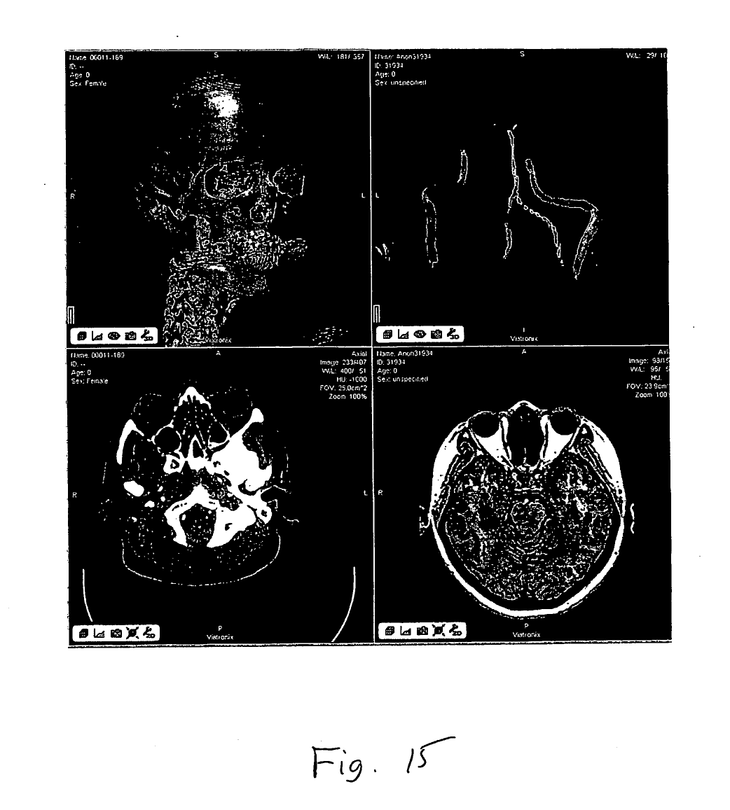

More specifically, the v3D Explorer has icons representing containers for the volume rendering settings. The user can drag and drop them between any two views that have the same type of setting (i.e. the volume data for any view, or the virtual camera only for 3D views). For instance, as shown in FIG. 15, having separate icons for each type of setting allows having an arrangement of 2×2 viewers in which the two on the left share one dataset and the two on the right share another dataset. The two on top can be 3D views sharing the same virtual camera. The two on the bottom can be 2D views and can share the same slice position.

The V3D Explorer can present the 3D volumetric image in two aspects: Parallel or Perspective. In the Perspective view the 3D image takes on a more natural appearance because the projections of the lines into the distance will eventually intersect, as train tracks appear to intersect at the horizon. Painters use perspective for a more lifelike and truer appearance. Parallel viewpoint, however, assumes the observer is at an infinite distance from the object, and so the lines run parallel and do not intersect in the distance. This viewpoint is most commonly used to make technical drawings. To toggle from perspective to parallel viewpoint in the 3D image, and back, the user could use, e.g., the C Key (for “Camera”) on the keyboard.

The Window/Level and Colormap Button, found in the lower left corner of each image, is used to load preset transfer functions, or reset the image back to its initial Window/Level. The Sculpting Buttons (tool bar 91, FIG. 5b) are used for Sculpting. “Sculpting” in medical imaging is much like conventional sculpting—it's an art. And just as the sculptor sees the image he wants to bring out in the marble and chips away want he doesn't want, the V3D Explorer allows the user to “chip” away at the volume data in the 3D image (the voxels) that the user does not want to include in a snapshot of the anatomical area. This feature is used in the same manner, and in conjunction with, the Lasso feature (described below) and Segmentation in general, the idea of which is to label the area inside or outside the selected zone. All sculpting actions result in a listing in the Annotations Pane.

As noted above, the annotations (measurement) module provides functions that allow a user to measure or otherwise annotate images. Annotations include imbedded markers and annotations that the user generates during the course of the examination. The annotations allows the user to add comments, notes, and remarks during the evaluation, and label Components. As noted above, the V3D Explorer treats measurements as annotations. By using Measurements, the user can add comments and remarks to each annotation made during the evaluation. These remarks, along with any values and/or statistics associated with the measurement, are displayed in the Annotations pane. For instance, FIGS. 25a and b illustrates measurement Annotations in an annotations pane. The measured length (in millimeters), angle, volume, etc., and the measurements associated number, are shown in the 2D image as well as Annotation pane listing.

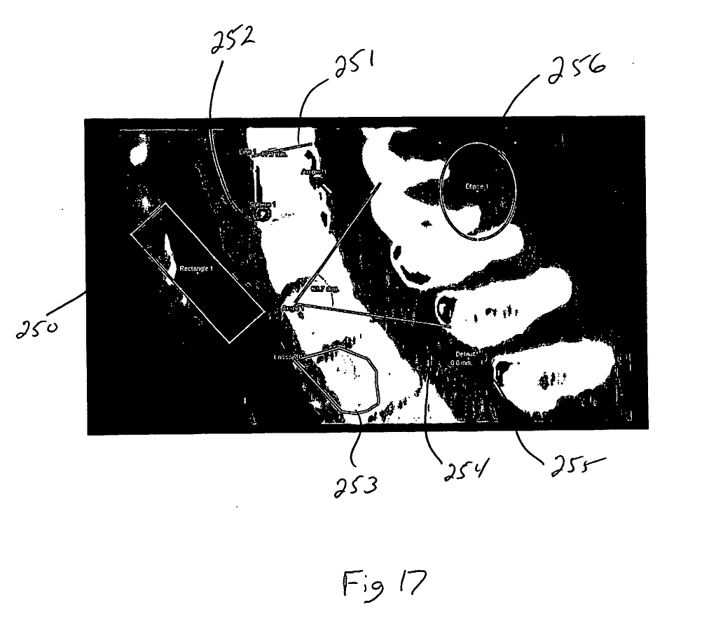

A “Linear” measurement button from the Tools button 91 is used to measure a straight line in the 2D slice images. Pressing the button 91 activates the linear measurement mode (which calculates the Euclidian distance between two points), and the mouse cursor changes shape. To measure, the user would place the cursor at the starting point, click the mouse, and drag the mouse to the next point. As the mouse move, one end point of the line stays fixed and the other moves to create the desired linear measurements. Releasing the mouse button draws a line and displays the length in millimeters (251, FIG. 17). The V3D Explorer automatically numbers the measurement for reference in case multiple measurements are made. Preferably, the accuracy of the linear measurement plus or minus one (1) voxel. Due to the resolution of the input scanner, the resolution of the length measurement is equivalent to the reconstructed “interslice distance.” The term “interslice distance” is used for the spacing between slices. Accuracy is determined in the other two planes (dimensions) by the scanner resolution unit, which is the spacing between the grid information (the voxels).

An “Angle” annotation tool from the User Tools 91 allows the user to draw two intersecting lines on the image and align them with regions of interest to measure the relative angle. This is a two step process, whereby first fix a point by clicking with the mouse, then extend the first leg of the triangle, and finally extend the second leg. A label and the angular measurement will be displayed (254, FIG. 17) and listed in the Annotations pane (243).

A Rectangle Annotation button creates a rectangle around a region of interest (250, FIG. 17), complete with a label, as the user holds the left mouse button down. The rectangle annotation can be adjusted using the “Adjust” annotation button.

An “Ellipse” annotation button provides a function similar to the rectangle annotation function except it generates an adjustable loop that the user can use to surround a region of interest (256, FIG. 17).

A freehand Selection Tool button (or alternatively referred to as “Lasso” or Region of Interest (ROI) tool) allows a user to encircle an abnormality, vessel, lesion or other area of interest with a “lasso” drawn with the mouse pointer (253, FIG. 17). After activating this feature, the user would hold down the left mouse button and the mouse pointer will change to represent a Freehand Selection tool. While holding down the left mouse button, use the mouse pointer to enclose the area you want to select. Lifting off of the mouse button will select the location.

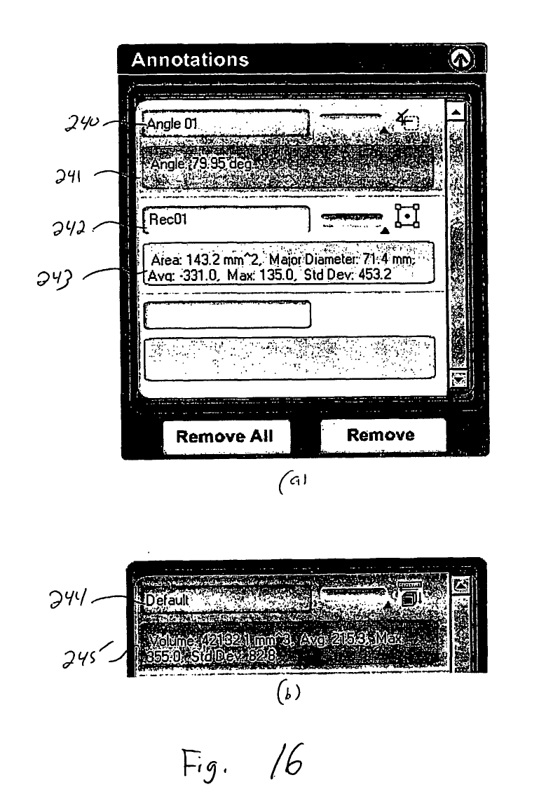

A Volume Annotation button can be selected to obtain the volume of a component. The Volume Annotation tool can only be performed on a previously defined component. Activating the Volume Annotation tool allows the user to click anywhere on a component (255. FIG. 17) and attain its volume, in cubed millimeters, average and maximum volumes, and the standard deviation. These values will be listed in the Annotation pane (as shown in FIGS. 16a and b, for example), and a label will be displayed on the image (“Default” is used until you change the label in the listing).

Various methods for generating the annotation and calculating the ROI statistics can be invoked to compute a histogram of the intensity distribution in the ROI and calculates the mean, maximum, minimum and standard deviation of the intensity within the ROI. Details of these methods are described in the above-incorporated provisional application.

Segmentation

Interactive segmentation allows a user to create, visualize, and adjust segmentation of any region within orthogonal, oblique, curved MPR slice images and 3D rendered images. Preferably, the interactive segmentation module uses an API to share the segmentation in all rendered views. The interactive segmentation module generates volume data to allow display of segmentation results and is interoperable with the measurement module to provide width, height, length, min, max, average, standard deviation, volume etc of segmented regions.

After the region grow process is finished, the associated volume or region of voxels are set as segmented volume data. The volume data is processed by the 2D/3D renderer to generate a 2D/3D view of the segmented component volume. The segmentation result are stored as component tag volume.

The user would select the “Segmentation” tool button in the User Tools Button bar (91, FIG. 5b). This button is used to toggle the Segmentation feature, and will open the Segmentation Pane (FIG. 8) when activated. The cursor will change to represent the segmentation tool, and the user will proceed to enter and display density threshold values. To create a new component, the user would first select the Input Intensity (121) option and then select the new (123) option in the add option box. Using the slider bars, the user would adjust the Low and the High density thresholds to desired values, or type the values directly into the Low and High boxes. Then, the user selects the display box to use these values high/low values and all areas and regions on the images corresponding to the threshold values will be visible. The user could then go to, e.g., a 2D view, axial slice, and click, which will select the entire component through all the slices and set a default color. The user could change the color if desired. To add another region to the component just defined, the user would click the Append box (124). The Append feature could be used until the component is completely defined. To define a new component, the user would select the New box (123) is checked, and repeat the above steps. Preferably, a dilate process is performed once after each segmentation process. To use the Sample Intensity feature (122) when in Segmentation mode, the user would click and check the Sample Intensity box (122). This will change the mouse pointer to the Segmentation Circle. The user would then move the circle over an area where the user wants to sample the threshold values. Click the left mouse button in that area if you want to use those values and select the component. The region will “grow” out from that point to every pixel having a density within the input threshold values.

Although the illustrative embodiments have been described herein with reference to the accompanying drawings, it is to be understood that the invention described herein is not limited to those precise embodiments, and that various other changes and modifications may be affected therein by one skilled in the art without departing from the scope or spirit of the invention. All such changes and modifications are intended to be included within the scope of the invention as defined by the appended claims.

1. A program storage device readable by machine, tangibly embodying a program of instructions executable by the machine to perform method steps for rendering a user interface for displaying medical images and enabling user interaction with the medical images, the method steps comprising:
displaying an image area that is divided into a plurality of views for viewing corresponding 2-dimensional and 3-dimensional images of an anatomical region; and
displaying a plurality of tool control panes that enable user interaction with the images displayed in the views, wherein plurality of tool control panes can be simultaneously opened and accessible.

2. The program storage device of claim 1, wherein the displayed tool control panes are arranged in a stack.

3. The program storage device of claim 1, further comprising instructions for automatically opening a plurality of control panes corresponding to a user interaction mode, in response to a user selection of the user interaction mode.

4. The program storage device of claim 1, wherein the control panes comprise a layouts pane that enables a user to select one of a plurality of layouts of the image area.

5. The program storage device of claim 1, wherein the control panes comprise a segmentation pane comprising a tool button that is selectable to automatically segment components of a displayed image within a user-specified intensity range.

6. The program storage device of claim 5, wherein the segmentation pane comprises a preset button that is selectable to automatically segment components of a displayed image within a predetermined intensity range.

7. The program storage device of claim 6, wherein the predetermined intensity range includes a range for air.

8. The program storage device of claim 6, wherein the predetermined intensity range includes a range for tissue.

9. The program storage device of claim 6, wherein the predetermined intensity range includes a range for muscle

10. The program storage device of claim 6, wherein the predetermined intensity range includes a range for bone.

11. The program storage device of claim 6, wherein the predetermined intensity range includes a user-specified range.

12. The program storage device of claim 5, wherein the control panes comprise a component pane that provides a list of segmented components.

13. The program storage device of claim 12, wherein the component pane comprises a tool button for locking a segmented component, wherein locking prevents the segmented component from being included in another segmented component during a segmentation process.

14. The program storage device of claim 12, wherein the component pane comprises an editable text field that enables a user to label a segmented component.

15. The program storage device of claim 12, wherein the component pane comprises a color selection button that enables a user to select a color in which the segmented component is displayed.

16. The program storage device of claim 15, wherein the component pane comprises a opacity selection button that enables a user to select an opacity for a selected color of the segmented component.

17. The program storage device of claim 12, wherein the component pane comprises a visibility selection button that enables a user to render a segmented component visible or invisible in a view.

18. The program storage device of claim 1, wherein the control panes comprise an annotations pane comprising a tool that enables acquisition and display of statistics of a segmented component.

19. The program storage device of claim 19, wherein the statistics comprise one of an average image intensity, a minimum image intensity, a maximum intensity, standard deviation of intensity, volume, and any combination thereof.

20. A program storage device readable by machine, tangibly embodying a program of instructions executable by the machine to perform method steps for rendering a user interface for displaying medical images and enabling user interaction with the medical images, the method steps comprising:
displaying an image area that is divided into a plurality of views for viewing corresponding 2-dimensional and 3-dimensional images of an anatomical region;
displaying icons representing containers for volume rendering settings, wherein volume rendering settings can be shared among a plurality of views or copied into another view.

21. The program storage device of claim 20, wherein a setting comprises volume data.

22. The program storage device of claim 20, wherein a setting comprises segmentation data.

23. The program storage device of claim 20, wherein a setting comprises a color map.

24. The program storage device of claim 20, wherein a setting comprises a window/level.

25. The program storage device of claim 20, wherein a setting comprises a virtual camera.

26. The program storage device of claim 20, wherein a setting comprises a 2D slice position.

27. The program storage device of claim 20, wherein a setting comprises a text annotation.

28. The program storage device of claim 20, wherein a setting comprises a position marker.

29. The program storage device of claim 20, wherein a setting comprises a direction marker.

30. The program storage device of claim 20, wherein a setting comprises a measurement annotation.

31. The program storage device of claim 20, wherein sharing is initiated by selecting a textual or graphical representation of the rendering setting and dragging the selected representation to a 2D or 3D view in which the selected representation is to be shared.

32. The program storage device of claim 31, wherein copying is performed by selection of an additional key while dragging the selected setting in the view.

33. A program storage device readable by machine, tangibly embodying a program of instructions executable by the machine to perform method steps for rendering a user interface for displaying medical images and enabling user interaction with the medical images, the method steps comprising:
displaying an image area that is divided into a plurality of views for viewing corresponding 2-dimensional (2D) and 3-dimensional (3D) images of an anatomical region; and
displaying an active 2D image in a 3D image to provide cross-correlation of the associated views.

34. The program storage device of claim 33, wherein the instructions for performing the step of displaying comprise instructions for rendering the 2D image in the 3D image with depth occlusion.

35. The program storage device of claim 33, wherein the instructions for performing the step of displaying comprise instructions for rendering the 2D image in the 3D view, wherein the 2D image is partially transparent.

36. The program storage device of claim 33, wherein the instructions for performing the step of displaying comprise instructions for rendering the 2D image as colored shadow on a surface of an object in the 3D image.

37. The program storage device of claim 33, comprising instructions for making the 2D image active by clicking on the associated 2D view.

38. The program storage device of claim 33, comprising instructions for making the 2D image active by moving a pointer over the 2D image view.
